(19) United States
(12) Patent Application Publication
(10) Pub. No.: US 2026/0183128 A1
(43) Pub. Date: Jul. 2, 2026

(54) RESPIRATION DRIVEN CAVAL OBSTRUCTION DEVICES AND METHODS

(71) Applicant: Edwards Lifesciences Corporation, Irvine, CA (US)

(72) Inventors: Yaeer E. Lev, Torrance, CA (US); Leonardo Paim Nicolau Da Costa, Tampa, FL (US); Allen Jeong Keel, Irvine, CA (US)

(21) Appl. No.: 19/545,963

(22) Filed: Feb. 20, 2026

Related U.S. Application Data
(63) Continuation of application No. PCT/US2024/046193, filed on Sep. 11, 2024.
(60) Provisional application No. 63/584,651, filed on Sep. 22, 2023.

(51) Int. Cl. A61F 2/48; A61B 17/12; A61F 2/06

(52) U.S. Cl. CPC A61F 2/484; A61B 17/12036; A61B 17/12109; A61B 17/12136; A61F 2002/068

(57) ABSTRACT

A system may include an outer frame. A system may include an expandable member, wherein the expandable is at least one or both of: disposed in the outer frame, and coupled to a lumen of the outer frame. A system may include a compressible bladder. A system may include a conduit configured to fluidly couple the expandable member to the compressible bladder, wherein forcing an inflation fluid from the compressible bladder to the expandable member is configured to manipulate the expandable member to a restricted blood flow state and forcing the fluid from the expandable member to the compressible bladder is configured to manipulate the expandable member to an unrestricted blood flow state.

### CROSS-REFERENCE TO RELATED APPLICATIONS

This application is a continuation of International Patent Application Ser. No. PCT/US 2024/046193, filed Sep. 11, 2024 and published Mar. 27, 2025 as WO2025064280, which claims the benefit of U.S. Provisional Patent Application Ser. No. 63/584,651, filed Sep. 22, 2023, the contents of each of which is herein incorporated by reference in its entirety.

### BACKGROUND

This disclosure relates generally to the field of medical devices and procedures, and more specifically to the field of blood flow management in blood vessels.

Patients with Heart Failure are often hospitalized due to increased pressures in the left atrium. The increased pressure in the left atrium is transmitted into the pulmonary circulation and ultimately leads to lung congestion and dyspnea. One mechanism that contributes to a patient's decompensation is volume redistribution from the splanchnic vascular network into circulation. Another mechanism may be an increase from normal levels of total blood volume (e.g., hypervolemia). Ultimately, the preload of the heart is increased to magnitude that it cannot keep up with.

### SUMMARY

In some aspects, the techniques described herein relate to a system for modulating blood flow through a blood vessel, the system including: an occluding device including: an outer frame, and an expandable member, wherein the expandable is at least one or both of: disposed in the outer frame, and coupled to a lumen of the outer frame; a compressible bladder; a conduit configured to fluidly couple the expandable member to the compressible bladder, wherein forcing an inflation fluid from the compressible bladder to the expandable member is configured to manipulate the expandable member to a restricted blood flow state and forcing the fluid from the expandable member to the compressible bladder is configured to manipulate the expandable member to an unrestricted blood flow state.

In some aspects, the techniques described herein relate to a system for modulating blood flow through a blood vessel, the system including: an occluding device including: an outer frame, a flow restricting device, wherein the flow restricting device is at least one or both of: disposed in the outer frame, and coupled to the outer frame; a compressible bladder; a mechanical linkage configured to couple the flow restricting device to the compressible bladder, wherein compression and decompression of the compressible bladder is configured to manipulate the flow restricting device.

In some aspects, the techniques described herein relate to a method of modulating blood flow through a blood vessel, the method including: implanting a flow modulating system in a patient, wherein the flow modulating device is configured to; increase occlusion of a blood vessel during inhalation of a patient; and decrease occlusion of the blood vessel during exhalation of the patient.

In some aspects, the techniques described herein relate to a method of modulating blood flow through a blood vessel, the method including: implanting a flow modulating system in a patient, wherein the flow modulating system is configured to; decrease occlusion of a blood vessel during inhalation of a patient; and increase occlusion of the blood vessel during exhalation of the patient.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates an embodiment of a flow modulating system including an occluding device positioned in a vessel.

FIG. 2 illustrates an embodiment of a flow modulating system including an occluding device positioned in a vessel.

FIG. 3A illustrates an embodiment of a flow restricting device in an unrestricted state.

FIG. 3B illustrates an embodiment of a flow restricting device in a partially restricted state.

FIG. 4 illustrates an embodiment of a flow modulating system including an occluding device positioned in the Superior Vena Cava of a patient and a compressible bladder positioned in the thoracic cavity of the patient.

FIG. 5 illustrates an embodiment of a flow modulating system including an occluding device positioned in the Superior Vena Cava of a patient and a compressible bladder positioned in the abdominal cavity of the patient.

FIG. 6 illustrates an embodiment of a flow modulating system including an occluding device positioned in the Inferior Vena Cava of a patient and a compressible bladder positioned in the thoracic cavity of the patient.

FIG. 7 illustrates an embodiment of a flow modulating system including an occluding device positioned in the Inferior Vena Cava of a patient and a compressible bladder positioned in the abdominal cavity of the patient.

FIG. 8 illustrates an embodiment of a method of modulating flow through a blood vessel.

FIG. 9 illustrates an embodiment of a method of modulating flow through a blood vessel.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiments. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel. The modulating and/or balancing of blood flow may be performed by the systems described herein to occlude, partially occlude, and/or otherwise manage, modulate, or regulate blood flow to or through a portion of a blood vessel. In some examples, such modulation and/or balancing of blood flow to or through a blood vessel may result in additionally modulating pressure in the right atrium of the heart and/or other organs of the body. The systems and methods may utilize anatomical forces to manipulate elements as described herein and, thus, may eliminate the need for electrical power sources.

The examples presented herein may relate to providing devices, methods, and/or methods of treatment (MOTs) for modulating and/or otherwise managing blood flow to or through one or more blood vessels. The terminology of restricting blood flow, regulating blood flow, modulating blood flow, managing blood flow, and balancing blood flow may be used to indicate regulating blood pressure, modulating blood pressure, managing blood pressure, and/or balancing blood pressure. As such, for example, a flow modulation device is synonymous with a pressure regulating device (i.e., a flow regulator is synonymous with a pressure regulator).

Managing blood flow through a blood vessel can be achieved by the devices and systems described herein to provide an advantage of providing a plurality of flow modulation states. For example, the flow modulating systems described herein can include one or more expandable members that can each be inflated to one of a plurality of inflation states to modulate flow through the flow modulating system and therefore in a vessel within which the device is installed or in a vessel in fluid communication with the vessel in which the device is installed. The amount of flow and/or pressure in a vessel can be tuned or modulated based on the inflation state of each expandable member of the one or more expandable members. A predetermined inflation state of an expandable member may be based on a blood pressure in the vessel, such that the pressure in the expandable member in the predetermined inflation state exceeds the blood pressure in the vessel. Additionally, or alternatively, a predetermined inflation state of an expandable member may be based on an inflation volume of the expandable member and/or a desired cross-sectional area reduction (or a desired cross-sectional area increase) of a cross-section of the lumen of an outer frame to which the expandable member is coupled. Additionally, or alternatively, the predetermined inflation state of the expandable member and/or cross-sectional area reduction of a cross-section of the lumen of an outer frame may be based on the magnitude of exhalation or inhalation performed by the patient. Alternative embodiments of the flow modulating systems described herein can include one or more flow restriction devices that can each be manipulated to one of a plurality of flow restriction states to modulate flow through the flow modulating system and therefore in a vessel within which the device is installed or in a vessel in fluid communication with the vessel in which the device is installed. A predetermined flow restriction state of a flow restricting device may be based on anatomical pressure in a cavity of a patient. Additionally, or alternatively, the predetermined flow restriction state of the flow restricting device may be based on the magnitude of exhalation or inhalation performed by the patient.

In addition, the devices, methods, and/or MOTs described herein can solve the technical problem of accumulation of blood in the venous system. For example, the devices and systems described herein may be used to reduce the accumulation of blood in the venous system, which can provide an advantage and technical effect of ensuring that pressure is not increased in the Inferior Vena Cava (IVC). Such devices can advantageously eliminate excessive hospital readmissions and/or can provide for a long-term blood flow management therapy, improving both quality of life and overall survival rates and with a lower cost to a healthcare system.

In some cases, patients who suffer from congestive heart failure (CHF) can also experience impaired renal function, as impaired renal function can be caused by increased systemic venous congestion as a result of low cardiac output and low blood pressure. The renal pressure gradient (between the renal arteries and renal veins) may be decreased due to elevated renal venous pressure, lowering glomerular filtration rate (GFR). GFR is the rate at which the kidney filters blood, for example, below 90 mL/min, which can be indicative of chronic kidney disease (CKD) that may eventually lead to end stage renal failure. Thus, reduction of renal venous pressure may improve GFR and reduce blood volume retention. Moreover, any such solution, when provided as an implantable system, can be percutaneously deliverable and can operate in a manner that minimizes risk of thrombosis.

In addition, the devices, methods, and/or MOTs described herein may function to advantageously limit, stagnate, and/or impede blood flow into the IVC from the hepatic veins to increase the pressure gradient between the IVC and the liver and/or splanchnic venous circulation. In some examples, one or more flow modulating devices or systems may be configured for placement at least partially within the hepatic veins and/or IVC and/or at one or more junctions between the hepatic veins and the IVC. As a result, blood flowing from the splanchnic venous reservoir into the hepatic veins can be slowed to increase blood volume in the splanchnic venous reservoir.

### SYSTEMS AND DEVICES

The systems and devices described herein function to modulate blood flow in a vessel. In some embodiments, the systems and devices described herein can function to reduce systemic venous congestion, reduce renal venous pressure, improve glomerular filtration rate (GFR), and/or reduce blood volume retention. The systems and devices are used for intravascular therapy, but can additionally, or alternatively, be used for any suitable applications, clinical or otherwise. The systems and devices can be configured and/or adapted to function for any suitable flow modulation function in a vessel.

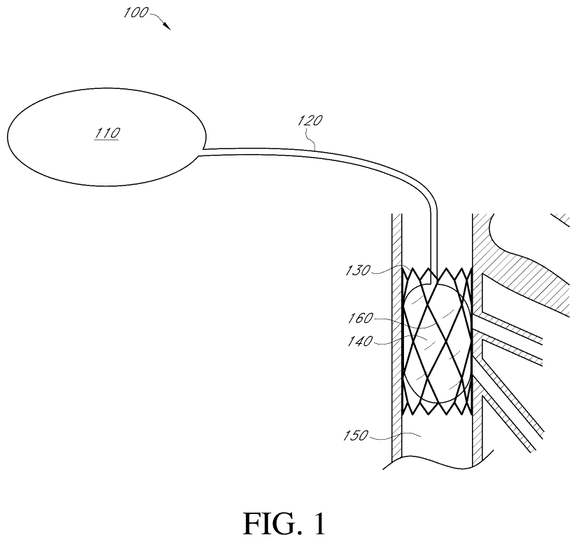

FIG. 1 illustrates an embodiment of a flow modulating system 100. The flow modulating system 100 may function to actuate the occluding device 160 by compressing or decompressing bladder 110. Actuating occluding device 160 may modulate flow in a vessel, or adjacent vessel, in which the occluding device 160 is installed. In some embodiments, as shown in FIG. 1, the flow modulating system 100 can include a compressible bladder 110, an occluding device 160, and a conduit 120 connecting (e.g., fluidly, mechanically, etc.) the compressible bladder to the occluding device 160. The occluding device 160 may include an outer frame 130 and an expandable member 140 disposed in the outer frame 130.

The occluding device 160 may be positionable within a blood vessel 150 (e.g., Superior Vena Cava, Inferior Vena Cava, etc.). In some embodiments, the expandable member 140 may be fluidly coupled (e.g., hydraulically, pneumatically, etc.) to the compressible bladder 110 by the conduit 120. Embodiments of the flow modulating systems described herein may be implanted in and actuated by an anatomy of a patient. The fluidly coupled flow modulating system 100 may contain an inflation fluid. Pneumatically actuated embodiments may utilize a compressible inflation fluid (i.e., a gas), for example, air (nitrogen-oxygen mixture), carbon dioxide, oxygen, helium, or any other appropriate gases known in the art. In use cases in which medical imaging (e.g., Magnetic Resonance Imaging) may be used as a visual aid for actuation confirmation or diagnostics, a low-density gas, such as helium, or the like, may be used to increase imaging contrast between the flow modulating system and the surrounding anatomy. Hydraulically actuated embodiments may utilize an incompressible inflation fluid (i.e., a liquid), for example, saline, water, contrast (e.g., iodine, barium, gadolinium), or any appropriate liquid known in the art. In use cases in which medical imaging (e.g., Magnetic Resonance Imaging) may be used as a visual aid for actuation confirmation or diagnostics, contrast may be used to increase imaging contrast between the flow modulating system and the surrounding anatomy.

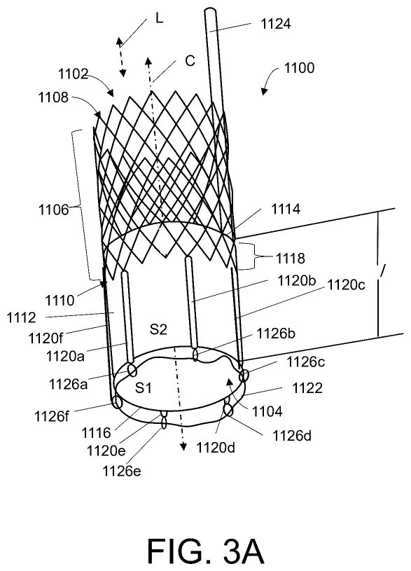

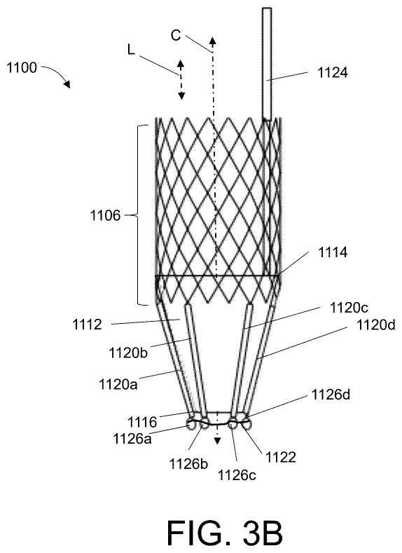

The flow modulating system 100 of FIG. 1, implanted in a patient 600 (shown in FIGS. 3-6), can be actuated (e.g., pneumatically, hydraulically, etc.) by the anatomical pressures of the patient in which the system 100 may be implanted. The compressible bladder 110 may be positioned in any portion of the patient anatomy which experiences regular variances in pressure. For example, the thoracic cavity 680, illustrated in FIGS. 3-6, experiences cyclical fluctuations in pressure during the breathing cycle of a patient. During inhalation, the thoracic cavity 680 expands, causing a decrease in pressure. Reducing the pressure to less than atmospheric pressure causes air to inflate the lungs. Conversely, during exhalation, the thoracic cavity 680 contracts, causing an increase in pressure that expels air from the lungs. Another example of patient anatomy, which experiences variances in pressure, is the abdominal cavity 690 illustrated in FIGS. 3-6. During inhalation, the abdominal cavity 690 constricts, causing an increase in pressure. Conversely, during exhalation, the abdominal cavity 690 expands, causing a decrease in pressure. As such, the compressible bladder 310 may be positioned in the thoracic cavity 680, the abdominal cavity 690, or any other portion of the anatomy in which pressure regularly fluctuates. The flow modulating system 100 of FIG. 1 may be configured such that the compressible bladder 110 is compressed during a period of increased pressure in the anatomy in which the compressible bladder 110 is implanted within, causing the compressible bladder 110 to drive inflation fluid into the expandable member 140 of the occluding device 160. The inflation fluid is driven from the compressible bladder to inflate the expandable member.

The occluding device 160 may be positioned in any bodily lumen or vessel (e.g., inferior vena cava, superior vena cava, renal artery, renal vein, etc.) to regulate flow therethrough or through an adjacent vessel fluidly connected to the vessel in which the occluding device 160 is positioned. The occluding device 160 may include an outer frame 130 having an inflow end 1102 (shown in FIG. 3A) and an outflow end 1104 (shown in FIG. 3A). The outer frame 130 (shown in FIG. 1) defines a lumen comprising an inner surface S1 between the inflow end 1102 and the outflow end 1104. The expandable member 140 may be disposed in and/or coupled to at least a portion of the inner surface S1 (shown in FIG. 3A) of the outer frame 130. In some embodiments, one or both of the inflow end 1102 or outflow end 1104 of the frame 130 may be tapered. The taper may further reduce blood flow through the vessel in addition to the occluding device 160 disposed in the frame 130. In some embodiments, the occluding device 160 may be coupled to the tapered end or ends of the frame 130 such that the taper of the frame 130 may be increased or decreased due to contraction or expansion of the occluding device 160, respectively.

The expandable member 140 includes a fluid connection or port to the conduit 120 for receiving an inflation fluid therethrough to inflate the expandable member 140. The expandable member 140 may define a volume configured to receive the inflation fluid therein through the conduit 120 to inflate the expandable member 140. The expandable member 140 is reversibly inflatable to a plurality of inflation states to partially or fully occlude the lumen of the outer frame 130. For example, a volume defined by the expandable member 140, in an unrestricted flow state, may be empty or have substantially no inflation fluid in the volume defined by the expandable member 140. Further, for example, a volume defined by the expandable member 140, in a restricted flow state, may include inflation fluid in the volume and/or be substantially full of an inflation fluid in the volume defined by the expandable member 140. Still further, for example, in an intermediate or partial flow restricted state, a volume defined by the expandable member may be partially full (or partially empty) such that an inflation fluid is used to partially fill the volume defined by the expandable member 140. Although a restricted flow state, an unrestricted flow state, and an intermediate flow state are described, one of skill in the art will appreciate that any number of intervening flow states between the aforementioned flow states is also possible and contemplated herein. Further, although an expandable member is described herein, one of skill in the art will appreciate that the expandable member may be one or more expandable members, more than one expandable member, or a plurality of expandable members such that the compressible bladder can inflate the one or more, more than one, or the plurality of expandable members.

Compressible bladders described herein may include re-expanding capabilities (i.e., shape memory properties). Said another way, if the pressure on a compressed compressible bladder is removed, or at least reduced, the volume of the compressible bladder may increase allowing inflation fluid from the expandable member to return into the compressible bladder. Additionally, one or more expandable members described herein may include self-constricting properties (i.e., elastic properties) such that the one or more expandable members reduce in volume when inflation pressure is removed. Said another way, if pressure is removed, or at least reduced, from the inflation fluid inflating the expandable member, the expandable member may constrict and force the internal inflation fluid therefrom. Embodiments of flow modulating systems described herein may include neither of, one of, or both of: a re-expanding compressible bladder, and self-constricting expandable member. Expandable members and compressible bladders without self-constricting properties or re-expanding properties are neither biased towards a restricted state or unrestricted state, but instead are manipulated to such states, or any state therebetween, by pressure. Embodiments of the flow modulating system 100 with neither a re-expanding compressible bladder, or self-constricting expandable member may be actuated based on the pressure differential between the pressure acting on the compressible bladder 110 and the pressure acting on the expandable member 140. The pressure differential between the pressure on the compressible bladder 110 and the expandable member 140 may be leveraged by embodiments described herein by increasing the volume of the compressible bladder 110 with respect to the volume of the expandable member 140. Increasing the volume of the compressible bladder 110 increases the amount of pressure and volume that the expandable member 140 experiences to equilibrate with a pressurized compressible bladder 110. Further, a flow modulating system 100 may include a compressible bladder with self-constricting properties (i.e., elastic properties). A flow modulating system 100 may include an expandable member with re-expanding properties (i.e., shape memory properties).

Flow modulating systems described herein, whether pneumatically actuated or hydraulically actuated, may have the volume or pressure of fluid contained by the system adjusted, for example, to achieve different levels of actuation, achieve calibration at different atmospheric pressure levels, or disable the flow modulating system by removing a portion or the entirety of the fluid therein. Fluid may be injected or withdrawn from the flow modulating system to achieve adjustment. Fluid may be injected into or withdrawn from the system implanted within a patient, for example, subcutaneously via a syringe.

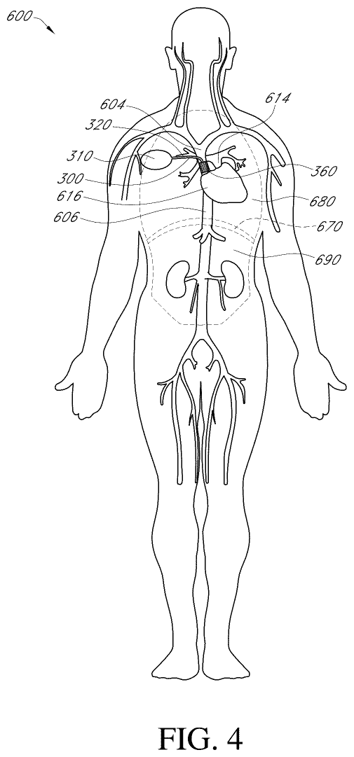

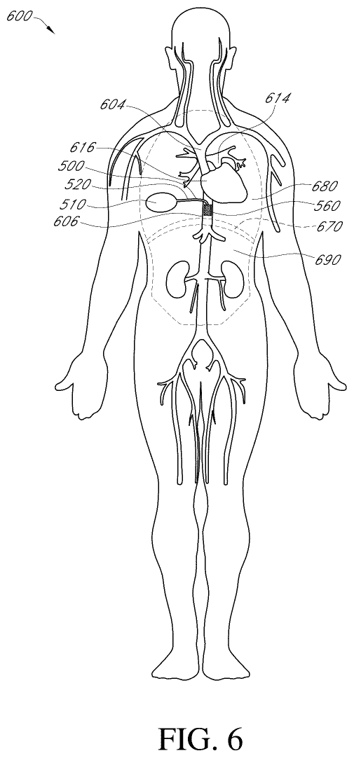

The flow modulating system 100 of FIG. 1 may be positioned and adapted to at least partially occlude any bodily lumen or vessel during exhalation of a patient 600 (shown in FIG. 4 and FIG. 6). As shown in FIG. 4, the compressible bladder 310 may be placed in the thoracic cavity 680 of the patient 600. As such, exhalation of the patient 600 causes an increase of pressure within the thoracic cavity 680, driving inflation fluid from the compressible bladder 310 to the expandable member of the occluding device 360. The driven inflation fluid inflates the expandable member of the occluding device 360 causing at least partial occlusion of the vessel in which the occluding device 360 is positioned within. When the patient 600 begins to inhale, pressure on the compressible bladder 310 is reduced, allowing the expandable member of the occluding device 360 to constrict and drive inflation fluid back to the compressible bladder 310. Additionally, or alternatively, the reinflation of the compressible bladder 310 may draw the inflation fluid from the expandable member of the occluding device 360 back into the compressible bladder 310. This cyclical process may continue indefinitely, increasing blood flow restriction through the blood vessel during exhalation, and reducing blood flow restriction through the blood vessel during inhalation. The aforementioned process may be performed by the flow modulating systems of both FIG. 4 and FIG. 6. FIG. 4 illustrates an embodiment of a flow modulating system 300 for modulating flow through the Superior Vena Cava 604, in which the occluding device 360 is positioned. FIG. 6 illustrates an embodiment of a flow modulating system 500 for modulating flow through the Inferior Vena Cava 606, in which the occluding device 560 is positioned.

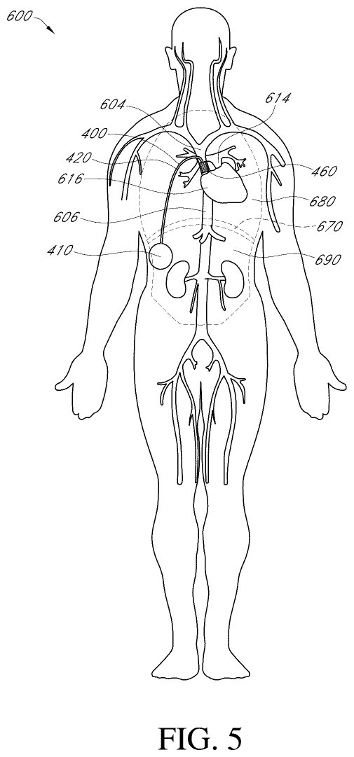

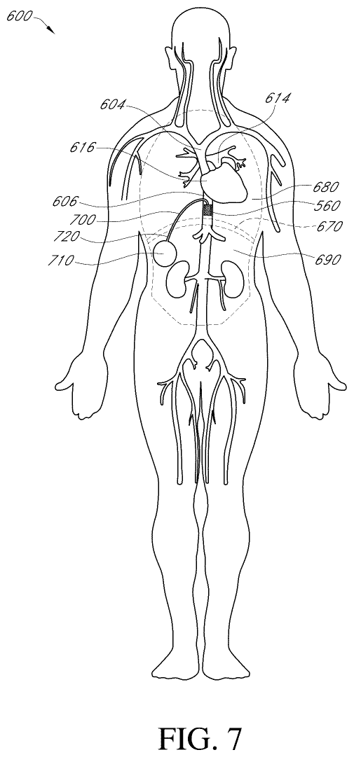

The flow modulating system 100 of FIG. 1 may be positioned and adapted to at least partially occlude any bodily lumen or vessel during inhalation of a patient 600 (shown in FIG. 5 and FIG. 7). As shown in FIG. 5, the compressible bladder 410 may be positioned in the abdominal cavity 690 of the patient 600. As such, inhalation of the patient 600 causes an increase of pressure within the abdominal cavity 690, driving inflation fluid from the compressible bladder 410 to the expandable member of the occluding device 460. The driven inflation fluid expands the expandable member of the occluding device 460 causing at least partial occlusion of the vessel in which the occluding device 460 is positioned within. When the patient 600 begins to exhale, pressure on the compressible bladder 410 is reduced, allowing the expandable member of the occluding device 460 to constrict and drive inflation fluid back to the compressible bladder 410. The cyclical process may continue indefinitely, increasing blood flow restriction through the blood vessel during inhalation, and reducing blood flow restriction through the blood vessel during exhalation. The aforementioned process may be performed by the flow modulating systems of both FIG. 5 and FIG. 7. FIG. 5 illustrates an embodiment of a flow modulating system 400 for modulating flow through the Superior Vena Cava 604, in which the occluding device 460 is positioned. FIG. 7 illustrates an embodiment of a flow modulating system 700 for modulating flow through the Inferior Vena Cava 606, in which the occluding device 760 is positioned.

Flow modulating systems described herein and shown in FIG. 1 may be adapted to perform in a large range of pressure differential scenarios. The pressure differentials are measured with respect to the pressures experienced upon the compressible bladder 110 and the pressures experienced upon the expandable member 140. For example, in achieving the appropriate actuation, the compressible bladder 110 and the expandable member 140 may be constructed of material with re-expanding properties or with self-constricting properties. In a first example scenario, the expandable member 140 may experience pressures larger than the pressures experienced by the compressible bladder 110. In the first example scenario, it may be advantageous to construct the expandable member 140 with a material having re-expanding properties and/or construct the compressible bladder 110 with a material having self-constricting properties. As such, the flow modulating system 100 of the first example scenario, may be biased in driving inflation fluid to the expandable member 140. In a second example scenario, the compressible bladder 110 may experience pressures larger than the pressures experienced by the expandable member 140. In the second example scenario, it may be advantageous to construct the expandable member 140 with a material having self-constricting properties and/or construct the compressible bladder 110 with a material having re-expanding properties. As such, the flow modulating system 100 of the second example scenario, may be biased in driving inflation fluid to the compressible bladder 110. Biasing the flow modulating systems may reduce the effects of the pressure differentials between the compressible bladder 110 and the expanding member 140, allowing proper actuation of the flow modulating systems described herein. Another example configuration of a flow modulating system 100 may include a compressible bladder 110 formed of one or more materials having self-constricting properties and an expandable member 140 formed of one or more materials having self-constricting properties. Another example configuration of a flow modulating system 100 may include a compressible bladder 110 formed of one or more materials having re-expanding properties and an expandable member 140 formed of one or more materials having re-expanding properties. Another example configuration of a flow modulating system 100 may include a compressible bladder 110 without self-constricting properties or re-expanding properties, and an expandable member 140 formed of one or more materials having self-constricting properties. Another example configuration of a flow modulating system 100 may include a compressible bladder 110 without self-constricting properties or re-expanding properties, and an expandable member 140 formed of one or more materials having re-expanding properties. Another example configuration of a flow modulating system 100 may include a compressible bladder 110 formed of one or more materials having self-constricting properties, and an expandable member 140 without self-constricting properties or re-expanding properties. Another example configuration of a flow modulating system 100 may include a compressible bladder 110 formed of one or more materials having re-expanding properties, and an expandable member 140 without self-constricting properties or re-expanding properties. Although one expandable member 140 is shown in FIG. 1, one of skill in the art will appreciate that more than one or a plurality of expandable members may be used in a flow modulating system 100.

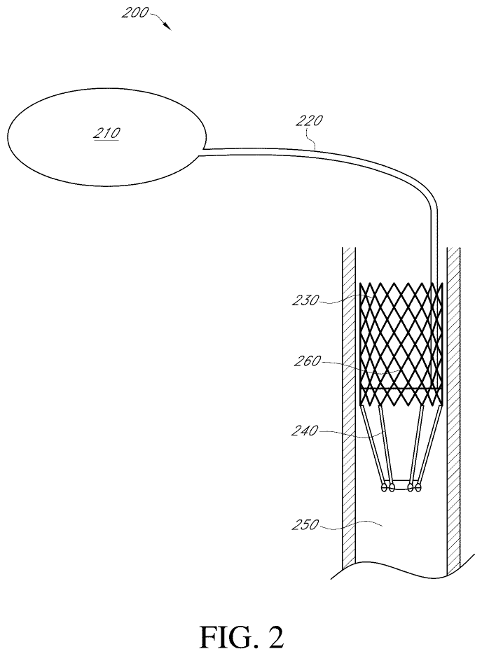

FIG. 2 illustrates another embodiment of a flow modulating system 200. The flow modulating system 200 may function to actuate the occluding device 260 by compressing or decompressing bladder 210. Actuating occluding device 260 may modulate flow in a vessel, or adjacent vessel, in which the occluding device 260 is installed. The flow modulating system 200 may include a mechanical linkage 220, an occluding device 260, and an optional compressible bladder 210. The occluding device 260 includes an outer frame 230 and a flow restricting device 240. The occluding device 260 is illustrated within a blood vessel 250.

In some embodiments, the flow restricting device 240 is mechanically linked to the compressible bladder 210 by the mechanical linkage 220 (e.g., a rigid push or pull member (e.g., a rod or rigid wire), a flexible tension member, a flexible wire, a suture, a string, a cable, a flexible elongate element, a rigid elongate element, a cable within a coil, etc.). Some embodiments of the flow modulating systems described herein may be implanted in and actuated by the anatomy of a patient.

In some mechanical-actuation embodiments that do not include a compressible bladder, a first end of the mechanical linkage 220 may be coupled to an intercostal region (e.g., rib) of the patient via a subcutaneous procedure. A second end of the mechanical linkage 220 may be connected to a control element (e.g., pullwire, cable, tether, etc.) of the occlusion device at either, for example, the jugular vein or femoral vein access point (depending on SVC or IVC location of the occlusion device). As the diaphragm contracts during inspiration, the chest wall and lungs expand, thereby expanding the intercostal region outward. The radial displacement of the intercostal region can result in translational motion of the mechanical linkage 220 and thus the control element of the occlusion device to pull the occlusion device towards a closed position. As the diaphragm relaxes and moves in a superior or cranial direction during exhalation, the chest cavity and lungs contract, and the inward radial displacement of the intercostal region results in translational motion of the mechanical linkage 220 to release tension force, allowing the occlusion device to reduce its occlusion of the vessel.

Further, in some mechanical-actuation embodiments that do not include a compressible bladder, a first end of the mechanical linkage 220 may be tethered to a superior or cranial side of a diaphragm, for example, via a subxiphoid puncture access approach. A second end of the mechanical linkage 220 may be connected to a control element (e.g., pullwire, cable, tether, etc.) of the occlusion device at either, for example, the jugular vein or femoral vein access point (depending on SVC or IVC location of the occlusion device). As the diaphragm contracts and moves in the caudal or inferior direction during inspiration, the displacement in the caudal or inferior direction results in translational motion of the control element to pull the occlusion device towards a closed, occluded, or at least partially occluded position. As the diaphragm relaxes and moves in a superior or cranial direction during expiration, the displacement in the cranial or superior direction results in translational motion of the control element to release tension force, allowing the occlusion device to open or allow increase blood flow therethrough.

FIG. 3A illustrates a bottom-up perspective view of an embodiment of an occluding device 1100 for modulating blood flow through a blood vessel. The occluding device 1100 may be actuated by the flow modulating system 200 of FIG. 2. In this embodiment, the device 1100 is shown in an unrestricted blood flow state. The unrestricted blood flow state may represent a state of device 1100 in which both an inflow end 1102 (e.g., an inflow) and an outflow end 1104 (an outflow) are open to receive fluid (e.g., blood, etc.). The fluid flows through the inflow end 1102 and through the device 1100 to the outflow end 1104 at least partially along an inner surface S1 opposite an outer surface S2 and through an opening defined by the device 1100. For example, the device 1100 may be in the expanded state when both the inflow end 1102 and the outflow end 1104 are open to receive fluid (e.g., blood, etc.) therethrough when the device 1100 is implanted in a blood vessel. The device 1100 includes an expandable frame 1106 that includes a proximal end 1108 and a distal end 1110, and a longitudinal axis (L) extending therethrough. The proximal end 1108 may correspond to the inflow end 1102 of the device 1100. The frame 1106 may be a stent, for example constructed of metal wire (e.g., stainless steel, platinum, Nitinol® wire or another shape memory alloy), or other material suitable for implantation in the human body. The device 1100 also includes a membrane (e.g., a flow restricting device) with an inflow end 1114 and an outflow end 1116. The inflow end 1114 is shown at least partially installed within the distal end 1110 of the expandable frame. For example, the membrane (e.g., a flow restricting device) is coupled to an inner surface portion of the expandable frame 1106, as shown by an overlap 1118. For example, the inflow end 1114 may be reversibly coupled to the distal end 1110 of the frame 1106. The membrane (e.g., a flow restricting device) may be formed of a polymer a copolymer, a textile (e.g., woven, knitted, nonwoven, or braided), a tissue (e.g., bovine pericardium, equine pericardium, porcine vena cava, etc.), or a combination thereof.

In some examples, the membrane (e.g., a flow restricting device) 1112 is adjustable to any number of positions between expanded and collapsed. For example, the outflow end 1116 may collapse inward toward the central axis (C) associated with the frame 1106 and at any interval between fully expanded and fully contracted. The outflow end 1116 may also open or expand outward away from the central axis (C) associated with the frame 1106. In some examples, the membrane (e.g., a flow restricting device) may be expanded or contracted from a particular device state into an expanded position, a partially expanded position, or a collapsed position. For example, when the device 1100 is in the expanded position, the device 1100 may be caused to be configured into a partially expanded position or a collapsed position by partially or fully collapsing, respectively, the outflow end 1116 of the membrane (e.g., a flow restricting device) toward the central axis (C). When the device 1100 is in the collapsed position, the device 1100 may be caused to be configured into a partially expanded position or an expanded position by partially or fully expanding, respectively, the outflow end 1116 of the membrane (e.g., a flow restricting device) toward the central axis (C). The expanded position of the membrane (e.g., a flow restricting device) may allow the blood flow through the blood vessel. For example, when the device 1100 is implanted in a blood vessel and is configured in the expanded position, the device 1100 may allow blood to flow from the inflow end 1114 through to the outflow end 1116, without substantially hindering the blood flow speed or the blood flow amount. The partially expanded position of the membrane (e.g., a flow restricting device) may allow partial occlusion of the blood vessel. For example, when the device 1100 is implanted in a blood vessel and is configured in the partially expanded position, the device 1100 may allow a partial amount of blood to flow from the inflow end 1114 through to the outflow end 1116 and may hinder a flow of the blood flow by a predefined amount associated with a cross sectional area formed when the outflow end 1116 is partially closed (e.g., partially collapsed, partially expanded). The collapsed position of the membrane (e.g., a flow restricting device) may occlude the blood vessel. In some embodiments, the occlusion of the blood vessel is a full occlusion. In some embodiments, the occlusion of the blood vessel is a partial occlusion.

As shown in FIG. 3A, the outflow end 1116 of the membrane (e.g., a flow restricting device) is coupled to a plurality of elongate support members 1120a, 1120b, 1120c, 1120d, 1120e, and 1120f. The elongate support members 1120a-1120f may be flexible to allow the membrane (e.g., a flow restricting device) to bend radially toward the central axis (C) of the frame 1106 at the outflow end of the membrane (e.g., a flow restricting device) when the control wire is actuated. As shown in FIG. 3A, the device 1100 further includes an eyelet 1126a, an eyelet 1126b, an eyelet 1126c, an eyelet 1126d, an eyelet 1126e, and an eyelet 1126f. The eyelets 1126a-1126f may be coupled to respective support members 1120a-1120f. For example, the eyelet 1126a is coupled to a distal end of the support member 1120a; the eyelet 1126b is coupled to a distal end of the support member 1120b; the eyelet 1126c is coupled to a distal end of the support member 1120c; the eyelet 1126d is coupled to a distal end of the support member 1120d; the eyelet 1126e is coupled to a distal end of the support member 1120e; the eyelet 1126f is coupled to a distal end of the support member 1120f. Each eyelet 1126a-1126f may be configured to receive a portion of the control wire 1122 threaded therethrough. The aperture of each respective eyelet 1126a-1126e is arranged to receive the control wire 1122 when threaded therethrough such that when the control wire 1122 is actuated, the eyelets 1126a-1126f move radially (e.g., cinching each eyelet together) toward the central axis (C) of the expandable frame 1106. For example, actuating the control wire 1122 reversibly cinches the membrane (e.g., a flow restricting device) toward the central axis (C) by bringing the eyelets 1126a-1126f together at the outflow end 1116 of the membrane (e.g., a flow restricting device) 1112 to occlude or partially occlude a blood vessel in which the device 1100 is implanted.

Referring again to FIG. 3A, the outflow end 1116 of the membrane (e.g., a flow restricting device) may correspond to the outflow end 1104 of device 1100. The outflow end 1116 of the membrane (e.g., a flow restricting device) may be triggered to radially collapse toward the central axis (C) associated with the frame 1106. For example, the outflow end 1116 of the membrane (e.g., a flow restricting device) may be configured to radially collapse inward at the outflow end 1116 by moving the plurality of support members 1120a-1120f and attached eyelets 1126a-1126f toward the central axis (C) or radially collapse outward at the outflow end 1116 by moving the plurality of support members 1120a-1120f and attached eyelets 1126a-1126f away from the central axis (C). The radial collapse or expansion may occur in response to an actuation of a control wire 1122. The control wire 1122 may be coupled to a portion of the membrane (e.g., a flow restricting device) or at least one of the elongate support members 1120a-1210f to trigger the expansion or the collapse.

FIG. 3B illustrates a side view of the example flow restricting device of FIG. 3A. In this example, the device 1100 is shown with the membrane (e.g., a flow restricting device) 1112 in a partially collapsed state. The partially collapsed state may represent a restricted blood flow state in which the membrane (e.g., a flow restricting device) radially collapses toward the central axis (C) of the frame 1106 to reduce (or stop) blood flow through the blood vessel. Such a state may allow for a partial flow of blood, for example, through a lumen associated with the membrane (e.g., a flow restricting device). FIG. 3A, by contrast depicts the device 1100 in an unrestricted blood flow state in which the membrane (e.g., a flow restricting device) is depicted radially expanded away from the central axis (C) of the expandable frame 1106 to allow blood to flow through the blood vessel in which device 1100 is implanted. Positioning the membrane (e.g., a flow restricting device) in the partially collapsed state (e.g., a restricted blood flow state) shown in FIG. 3B, the control wire 1122 may be actuated by a control element 1124 coupled to, or otherwise in communication with, the control wire 1122 to cause tensioning of the control wire 1122 and closure or partial closure (e.g., cinching) of the membrane (e.g., a flow restricting device) at the outflow end 1116. For example, the membrane (e.g., a flow restricting device) may be adjustable to form a cinched portion at the outflow end 1116. The flow modulating system 200 of FIG. 2 may manipulate the membrane (e.g., a flow restricting device) 1112 of the occluding device 1100 of FIGS. 3A and 3B to any number of positions between, and including, expanded and collapsed. Manipulation of the occluding device 1100 may be performed by coupling the mechanical linkage 220 (shown in FIG. 2) to the control element 1124 (shown in FIGS. 3A and 3B). As such, actuation of the mechanical linkage 220 (shown in FIG. 2), as described herein, may cause tensioning and release of the control wire 1122 (shown in FIGS. 3A and 3B) to achieve any number of positions between, and including, expanded and collapsed.

The flow modulating system 200 of FIG. 2, positioned in a patient 600 (shown in FIGS. 3-6), may be actuated by the anatomical pressures of the patient in which the system 200 is positioned. The compressible bladder 210 may be positioned in any portion of the patient anatomy which experiences regular variances in pressure. For example, the thoracic cavity 680, illustrated in FIGS. 3-6, experiences cyclical fluctuations in pressure during the breathing cycle of a patient. Another example of patient anatomy which experiences regular variances in pressure, is the abdominal cavity 690, illustrated in FIGS. 3-6. As such, the compressible bladder 210 may be positioned in the thoracic cavity 680, the abdominal cavity 690, or any other portion of the anatomy in which pressure regularly fluctuates. The flow modulating system 200 of FIG. 2 may be configured in such a way that when the compressible bladder 210 is compressed during a period of increased pressure in the anatomy in which the compressible bladder 210 is positioned, the compressible bladder 210 can deform and manipulate the mechanical linkage 220. Deforming the compressible bladder 210 results in pushing or tensioning the mechanical linkage 220 attached to the flow restricting device 240 of the occluding device 260. Removing pressure from the compressible bladder 210 may allow the compressible bladder to expand (i.e., return to or toward an unmanipulated shape) from decompression and produce the opposite manipulation effect on the mechanical linkage 220. For example, if the compressible bladder 210 tensions the mechanical linkage 220 when compressed, the compressible bladder 210 may push the mechanical linkage 220 when decompressed. The flow restricting device 240 may restrict or un-restrict flow when actuated by the relaxing or tensioning of the mechanical linkage 220. The flow modulating system 200 illustrated in FIG. 2 includes a flow restricting device 240 which increases flow restriction when pulled by the mechanical linkage 220, but any appropriate flow restricting devices actuated by pushing and/or tensioning may be utilized in embodiments contemplated herein.

The flow modulating system 200 of FIG. 2 may be positioned and adapted to at least partially occlude any bodily lumen or vessel during exhalation of a patient 600 (shown in FIG. 4 and FIG. 6). As shown in FIG. 4, the compressible bladder 310 may be placed in the thoracic cavity 680 of the patient 600. As such, exhalation of the patient 600 causes an increase of pressure within the thoracic cavity 680, deforming (i.e., compressing) the compressible bladder 310, tensioning mechanical linkage 220 connected to the flow restricting device 240 (shown in FIG. 2), and causing increased flow restriction through the occluding device 360. For example, manipulation of the occluding device 1100 (shown in FIGS. 3A-3B) may be performed by coupling the mechanical linkage 220 (shown in FIG. 2) to the control element 1124 (shown in FIGS. 3A and 3B). As such, actuation of the mechanical linkage 220 (shown in FIG. 2), as described herein, may cause or release tension on the control wire 1122 (shown in FIGS. 3A and 3B) to modulate flow. When the patient 600 begins to inhale, pressure on the compressible bladder 310 is reduced, allowing the compressible bladder 310 to decompress or expand, reducing tension on the mechanical linkage 220 connected to the flow restricting device 240 (shown in FIG. 2), and reducing flow restriction through the occluding device 360. The cyclical process may continue indefinitely, increasing blood flow restriction through the blood vessel during exhalation, and reducing blood flow restriction through the blood vessel during inhalation. The aforementioned process may be performed by the flow modulating systems of both FIG. 4 and FIG. 6. FIG. 4 illustrates an embodiment of a flow modulating system 300 for modulating flow through the Superior Vena Cava 604, in which the occluding device 360 is positioned. FIG. 6 illustrates an embodiment of a flow modulating system 500 for modulating flow through the Inferior Vena Cava 606, in which the occluding device 560 is positioned.

The flow modulating system 200 of FIG. 2 may be positioned and adapted to at least partially occlude any bodily lumen or vessel during inhalation of a patient 600 (shown in FIG. 5 and FIG. 7). As shown in FIG. 5, the compressible bladder 410 may be placed in the abdominal cavity 690 of the patient 600. As such, inhalation of the patient 600 causes an increase of pressure within the abdominal cavity 690, deforming the compressible bladder 310, tensioning mechanical linkage 220 connected to the flow restricting device 240 (shown in FIG. 2), and causing increased flow restriction through the occluding device 360. When the patient 600 begins to inhale, pressure on the compressible bladder 310 is reduced, allowing the compressible bladder 310 to decompress or expand, reducing tension on the mechanical linkage 220 connected to the flow restricting device 240 (shown in FIG. 2), and reducing flow restriction through the occluding device 360. The aforementioned process may be performed by the flow modulating systems of both FIG. 5 and FIG. 7. FIG. 5 illustrates an embodiment of a flow modulating system 400 for modulating flow through the Superior Vena Cava 604, in which the occluding device 460 is positioned. FIG. 7 illustrates an embodiment of a flow modulating system 700 for modulating flow through the Inferior Vena Cava 606, in which the occluding device 760 is positioned.

Alternatively, embodiments of the flow modulating system 200 of FIG. 2 may be adapted to include a compressible bladder 210 that, when deformed by increased pressure, releases tension on the mechanical linkage 220, and causes a reduction in flow restriction. Such embodiments would increase flow restriction when pressure is removed, or at least reduced, from the compressible bladder 210. Further, embodiments of the flow modulating system 200 may utilize an occluding device 260 which reduces flow restriction when the flow restricting device 240 is tensioned by the mechanical linkage and increases flow restriction when tension on the flow restricting device 240 from the mechanical linkage 220 is reduced.

Mechanical linkages for flow modulating systems described herein may include cables, wires, flexible elongate elements, rigid elongate elements, a cable/wire within a coil, or any other appropriate elements known in the art for transferring force from the compressible bladder to the flow restricting device. Some flow modulating system embodiments contemplated herein utilize anatomical features for the actuation of the mechanical linkage. Said another way, these flow modulating system embodiments, instead of using a compressible bladder, may couple a mechanical linkage to a body part which regularly moves, for example, the rib of a rib cage. A flow modulating system with an occluding device positioned within a blood vessel may include a length dimension between a rib of a rib cage and the occluding device that regularly increases and decreases, for example, during inhalation and exhalation. The travel of the rib with respect to the occluding device may be used to manipulate the mechanical linkage for flow modulating processes described herein. For example, during inhalation the rib may pull on the mechanical linkage, and during exhalation the rib may release tension or push the mechanical linkage. The described manipulation of the mechanical linkage may be used to actuate the occluding device for flow modulation of the blood vessel.

Compressible bladders described herein may include a bistable mechanism for binary control of an occluding device. Flow modulating system embodiments with compressible bladders that do not include bistable mechanisms include proportional control of flow restriction. Said another way, flow modulating system embodiments with compressible bladders that do not include bistable mechanisms may include proportional flow restriction response from the occluding device with respect to pressure on the compressible bladder. For example, the flow modulating system 100 of FIG. 1 may proportionally increase flow restriction as pressure is increased on the compressible bladder 110, at least for a period of time. Further, and inversely, the flow modulating system may proportionally decrease flow restriction as pressure is decreased on the compressible bladder, at least for a period of time. A bistable mechanism, for example a convex disc made from an alloy, composite, plastic, or any other material with appropriate properties, is a mechanism that can withstand pressure until a predefined threshold is met or crossed. If a pressure greater than the pre-determined threshold pressure is experienced by the bistable mechanism, the bistable mechanism changes from a first shape to second shape (i.e., flips from convex to concave in the case of the convex disc) in a rapid manner (e.g., snap action). If the compressible bladder 110 includes a bistable mechanism (e.g., convex disc), the compressible bladder 110 under any pressure less than the threshold pressure may not transmit inflation fluid to the expandable member 140. If the pressure acting on the compressible bladder 110 is at or above the threshold pressure, the compressible bladder 110 can snap and actuate the expandable member 140 from a least flow restrictive state to a most restrictive state. The bistable mechanism actuated from a first shape to a second shape by a pressure greater than or equal to the threshold pressure may be held in the second shape by a hold pressure less than the threshold pressure. As such, the period of the most flow restrictive state of the flow modulating system may be determined by the threshold pressure and the differential between the threshold pressure and the hold pressure. In some embodiments, the hold pressure and the threshold pressure of the bistable mechanism are equal. If the pressure on the compressible bladder 110 becomes less than or equal to the hold pressure, the bistable mechanism snaps back to the first shape allowing inflation fluid to transfer back into the compressible bladder 110 and allowing the expandable member 140 to constrict back to the least flow restrictive state. Although described for the flow modulating system 100 embodiment of FIG. 1, the bistable mechanism may be utilized by the flow modulating system 200 of FIG. 2. The compressible bladder 210, in the same described manner, may snap from an undeformed (i.e., expanded or decompressed) first shape to a fully-deformed second shape. As such, the mechanical linkage 220 may move the flow restricting device 240 from a least flow restrictive state to a most flow restrictive state and may move the flow restricting device 240 from the most flow restrictive state to the least flow restrictive state. The snap action of a compressible bladder utilizing a bistable mechanism allows a flow modulating system to control flow restriction in a binary fashion. Said another way, the flow modulating system maintains flow at either a least restricted state or a most restricted state.

Compressible bladders described herein may be implanted such that the compressible bladders are fixed and stable. Compressible bladders may be attached (e.g., sutured) to anatomy of the patient, resulting in a stable and fixed coupling of the compressible bladders. Further, mechanical linkages and/or conduits described herein, for coupling (e.g., mechanically or fluidly) compressible bladders to occluding devices described herein, may be tunneled through the anatomy of a patient.

In some variations, the occluding device may include more than one expandable member for modulating flow through a vessel. In some variations, the expandable member can include an expandable balloon. In some embodiments, the expandable member and compressible bladder may include or comprise a compliant material. In some embodiments, the expandable member and compressible bladder may be formed of a compliant material. In some embodiments, the expandable members and compressible bladder may consist essentially of a compliant material. A compliant material may exhibit a burst pressure of about 0 atmospheres (atm) to about 2 atm. In some embodiments, a compliant material may be able to expand about 20% to about 500%. Non-limiting examples of compliant materials include silicones, latex, polyvinyl chloride, polyolefin copolymer, or a combination thereof.

In some instances, the expandable member and compressible bladder may include or comprise a semi-compliant material. In some embodiments, the expandable member and compressible bladder may be formed of a semi-compliant material. In some embodiments, the expandable member and compressible bladder may consist essentially of a semi-compliant material. A semi-compliant material may exhibit a burst pressure of about 1 atm to about 25.5 atm. In some embodiments, a semi-compliant material may be able to expand about 10% to about 20%. Non-limiting examples of semi-compliant materials include polyethylene terephthalate, nylons, thermoplastic polyurethanes, thermoplastic elastomers, or a combination thereof.

In some variations, the outer frame is an intraluminal device, a stent, a braided tubular or ovular structure, or the like. The outer frame can include one or more coatings or coverings thereon. For example, at least a portion of the outer frame may be covered in or coated in a polymer, a biomaterial, a textile, a drug, or the like. Further, for example, at least a portion of the outer frame can include one or more layers of material to facilitate coupling the expandable member to the outer frame.

In some embodiments, the outer frame, the occluding device, and/or the compressible bladder may include an embedded radiopaque marker. The use of the embedded radiopaque marker may increase visibility of the system elements using fluoroscopy during occluding device placement in a vessel, in repositioning the occluding device in a vessel, in extracting a device from a vessel, and/or in routine maintenance or check-ups on the system and/or the patient.

In some embodiments, any of the foregoing embodiments or mechanisms may be employed with other flow modulating systems to maintain the flow modulating systems in motion to reduce thrombosis development.

### METHODS

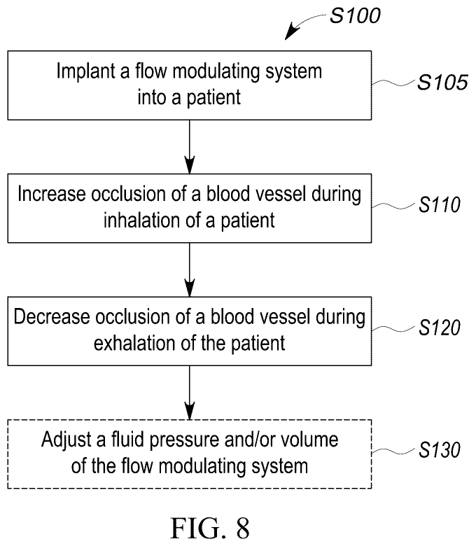

FIG. 8 illustrates a method S100 for modulating flow through a blood vessel. The method S100 includes: implanting a flow modulating system in a patient at block S105; such that the flow modulating system is configured to: increase occlusion of a blood vessel during inhalation of a patient at block S110; and decrease occlusion of a blood vessel during exhalation of the patient at block S120. The method S100 may include an optional step of adjusting a fluid pressure and/or volume of the flow modulating system in block S130. The method S100 functions to modulate flow through a blood vessel of a patient. The methods described herein are used for the medical field, but can additionally, or alternatively, be used for any suitable applications.

As shown in FIG. 8 method S100 for modulating flow through a blood vessel includes block S105, which recites implanting a flow modulating system in a patient. Appropriate flow modulating systems may be any properly configured flow modulating system described herein. Method S100 further includes block S110, which recites increasing occlusion of a blood vessel during inhalation of a patient. Block S110 may be performed by the flow modulating system 100 embodiment of FIG. 1 and the flow modulating system 200 embodiment of FIG. 2 configured for such, as described herein, to increase flow restriction performed by the occluding device during inhalation. Method S100 further includes block S120, which recites decreasing occlusion of a blood vessel during exhalation of the patient. Block S120 may be performed by the flow modulating system 100 embodiment of FIG. 1 and flow modulating system 200 embodiment of FIG. 2 configured for such, as described herein, to decrease flow restriction performed by the occluding device during exhalation. For example, the flow modulating system 100 of FIG. 1 (with the compressible bladder 110 implanted in the abdominal cavity) may increase occlusion during inhalation and decrease occlusion during exhalation. Such that inhalation compresses the compressible bladder 110, inflating the expandable member 140 of the occluding device 160, and exhalation decompresses the compressible bladder 110, deflating the expandable member 140 of the occluding device 160. The flow modulating system 100 of FIG. 1 may be implanted such that the compressible bladder 110 is fixed and/or stable (e.g., sutured to the anatomy, etc.), and the conduit 120 may be tunneled through the anatomy between the compressible bladder 110 and the occluding device 160. Optional block S130 recites adjusting a fluid pressure and/or volume of the flow modulating system. At any point of method S100, the fluid pressure and/or volume of the modulating system may be adjusted, for example, prior to, during, or after implantation within a patient. Fluid may be injected into or withdrawn from the flow modulating device, for example, subcutaneously via a syringe. Block S130 provides the ability to adjust the flow modulating system to, for example, achieve different levels of actuation, achieve calibration at different atmospheric pressure levels, or to disable the flow modulating system by removing a portion or the entirety of the fluid therein.

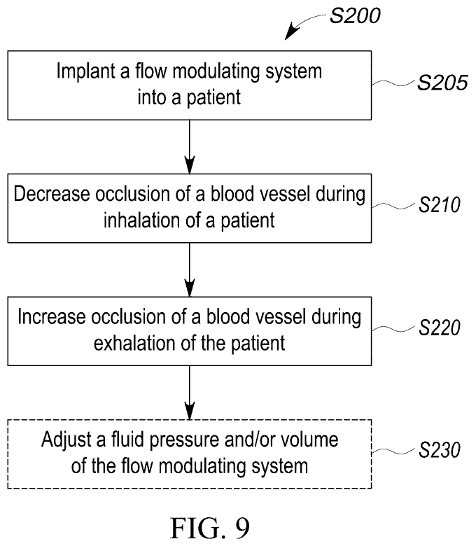

FIG. 9 illustrates a method S200 for modulating flow through a blood vessel. The method S200 includes: implanting a flow modulating system in a patient at block S205; such that the flow modulating system is configured to: decrease occlusion of a blood vessel during inhalation of a patient at block S210; and increase occlusion of a blood vessel during exhalation of the patient at block S220. The method S200 may include an optional step of adjusting a fluid pressure and/or volume of the flow modulating system in block S230. The method S200 functions to modulate flow through a blood vessel.

As shown in FIG. 9 method S200 for modulating flow through a blood vessel includes block S205, which recites implanting a flow modulating system into a patient. Appropriate flow modulating systems may be any properly configured flow modulating system described herein. The method S200 further includes block S210, which recites decreasing occlusion of a blood vessel during inhalation of a patient. Block S210 may be performed by the flow modulating system 100 embodiment of FIG. 1 and flow modulating system 200 embodiment of FIG. 2 configured for such, as described herein, to decrease flow restriction performed by the occluding device during inhalation. Method S200 further includes block S220, which recites increasing occlusion of a blood vessel during exhalation of the patient. Block S220 may be performed by the flow modulating system 100 embodiment of FIG. 1 and flow modulating system 200 embodiment of FIG. 2 configured for such, as described herein, to increase flow restriction performed by the occluding device during exhalation. For example, the flow modulating system 100 of FIG. 1 (with the compressible bladder 110 implanted in the thoracic cavity) may increase occlusion during exhalation and decrease occlusion during inhalation. Such that exhalation compresses the compressible bladder 110, inflating the expandable member 140 of the occluding device 160, and inhalation decompresses the compressible bladder 110, deflating the expandable member 140 of the occluding device 160. The flow modulating system 100 of FIG. 1 may be implanted such that the compressible bladder 110 is fixed and/or stable (e.g., sutured to the anatomy, etc.), and the conduit 120 may be tunneled through the anatomy between the compressible bladder 110 and the occluding device 160. Optional block S230 recites adjusting a fluid pressure and/or volume of the flow modulating system. At any point of method S200, the fluid pressure and/or volume of the modulating system may be adjusted, for example, prior to, during, or after implantation within a patient. Fluid may be injected into or withdrawn from the flow modulating device, for example, subcutaneously via a syringe. Block S230 provides the ability to adjust the flow modulating system to, for example, achieve different levels of actuation, achieve calibration at different atmospheric pressure levels, or to disable the flow modulating system by removing a portion or the entirety of the fluid therein.

The method S100 of FIG. 8 and the method S200 of FIG. 9 may be repeated cyclically based on the respiration of a patient. Furthermore, the amount of flow restriction of the method S100 of FIG. 8 and the method S200 of FIG. 9 may be based on the magnitude of breaths taken and released by a patient.

The devices and methods described herein may be used to treat a subject having any combination of or any one or more of heart failure, chronic kidney disease, chronotropic incompetence, inability to increase stroke volume, and/or peripheral microvascular dysfunction. In addition, the devices and methods described herein may be used for a method of treatment to regulate pressure in the right atrium of the heart. Further, the devices and methods described herein may be used for a method of treatment to improve function of the kidneys in patients having reduced kidney function due to pressure in the venous system.

For example, any of the implantable devices and/or systems described herein may be configured to modulate a volume of blood flowing from a superior vena cava into a right atrium to decrease right atrial pressure. Further for example, any of the implantable devices and/or systems described herein may be used to perform a method including restricting blood flow within a blood vessel. Still further for example, any of the implantable devices and/or systems described herein may be used to perform a method of treatment for a subject having one or both of: congestive heart failure or chronic kidney disease. The method may include restricting blood flow within the blood vessel.

### EXAMPLES

Example 1. A system for modulating blood flow through a blood vessel, the system comprising: an occluding device comprising: an outer frame, and an expandable member, wherein the expandable is at least one or both of: disposed in the outer frame, and coupled to a lumen of the outer frame; a compressible bladder; a conduit configured to fluidly couple the expandable member to the compressible bladder, wherein forcing an inflation fluid from the compressible bladder to the expandable member is configured to manipulate the expandable member to a restricted blood flow state and forcing the fluid from the expandable member to the compressible bladder is configured to manipulate the expandable member to an unrestricted blood flow state.

Example 2. The system of example 1, wherein the inflation fluid comprises a gas.

Example 3. The system of any one of the preceding examples, but particularly example 1, wherein the inflation fluid comprises a liquid.

Example 4. The system of any one of the preceding examples, but particularly example 1, wherein the compressible member comprises a bistable mechanism.

Example 5. The system of any one of the preceding examples, but particularly example 1, wherein the expandable member is configured to inflate during manipulation to the restricted blood flow state.

Example 6. The system of any one of the preceding examples, but particularly example 1, wherein the expandable member is configured to constrict during manipulation to the unrestricted blood flow state.

Example 7. The system of any one of the preceding examples, but particularly example 1, wherein the compressible bladder is configured to be positioned in a thoracic cavity of a patient.

Example 8. The system of any one of the preceding examples, but particularly example 1, wherein the compressible bladder is configured to be positioned in an abdominal cavity of a patient.

Example 9. The system of any one of the preceding examples, but particularly example 7, wherein the compressible bladder is configured to be compressed by an exhalation of the patient.

Example 10. The system of any one of the preceding examples, but particularly example 8, wherein the compressible bladder is configured to be compressed by an inhalation of the patient.

Example 11. The system of any one of the preceding examples, but particularly example 1, wherein the system does not comprise an electric power source.

Example 12. A method of treatment for a subject having one or both of: congestive heart failure or chronic kidney disease, comprising using the flow modulating system of any one of claims 1-11 to restrict blood flow within the blood vessel.

Example 13. A method of modulating blood flow within a blood vessel, comprising using the flow modulating system of any one of examples 1-11 to modulate blood flow within the blood vessel.

Example 14. A system for modulating blood flow through a blood vessel, the system comprising: an occluding device comprising: an outer frame, a flow restricting device, wherein the flow restricting device is at least one or both of: disposed in the outer frame, and coupled to the outer frame; a compressible bladder; a mechanical linkage configured to couple the flow restricting device to the compressible bladder, wherein compression and decompression of the compressible bladder is configured to manipulate the flow restricting device.

Example 15. The system of any one of the preceding examples, but particularly example 14, wherein the compressible bladder comprises a gas.

Example 16. The system of any one of the preceding examples, but particularly example 14, wherein the compressible bladder comprises a liquid.

Example 17. The system of any one of the preceding examples, but particularly example 14, wherein compression of the compressible bladder is configured to manipulate the flow restricting device to an increased flow restriction state and decompression of the compressible bladder is configured to manipulate the flow restricting device to a decreased flow restriction state.

Example 18. The system of any one of the preceding examples, but particularly example 14, wherein decompression of the compressible bladder is configured to manipulate the flow restricting device to an increased flow restriction state and compression of the compressible bladder is configured to manipulate the flow restricting device to a decreased flow restriction state.

Example 19. The system of any one of the preceding examples, but particularly example 14, wherein the compressible bladder comprises a bistable mechanism.

Example 20. The system of any one of the preceding examples, but particularly example 14, wherein the compressible bladder is configured to be positioned in a thoracic cavity of a patient.

Example 21. The system of any one of the preceding examples, but particularly example 20, wherein the compressible bladder is configured to be compressed by an exhalation of the patient.

Example 22. The system of any one of the preceding examples, but particularly example 14, wherein the compressible bladder is configured to be positioned in an abdominal cavity of a patient.

Example 23. The system of any one of the preceding examples, but particularly example 21, wherein the compressible bladder is configured to be compressed by inhalation of the patient.

Example 24. The system of any one of the preceding examples, but particularly example 14, wherein the system does not comprise an electric power source.

Example 25. The system of any one of the preceding examples, but particularly example 14, wherein the mechanical linkage is a cable.

Example 26. A method of treatment for a subject having one or both of: congestive heart failure or chronic kidney disease, comprising using the flow modulating system of any one of examples 14-25 to restrict blood flow within the blood vessel.

Example 27. A method of modulating blood flow within a blood vessel, comprising using the flow modulating system of any one of examples 14-25 to modulate blood flow within the blood vessel.

Example 28. A method of modulating blood flow through a blood vessel, the method comprising: implanting a flow modulating system in a patient, wherein the flow modulating device is configured to; increase occlusion of a blood vessel during inhalation of a patient; and decrease occlusion of the blood vessel during exhalation of the patient.

Example 29. A method of modulating blood flow through a blood vessel, the method comprising: implanting a flow modulating system in a patient, wherein the flow modulating system is configured to; decrease occlusion of a blood vessel during inhalation of a patient; and increase occlusion of the blood vessel during exhalation of the patient.

Example 30. The method of example 28 or example 29, further comprising adjusting a fluid pressure and/or volume of the flow modulating system.

As used herein, inhalation and inspiration may be used interchangeably to mean the process of bringing air from outside the body into the lungs. It is carried out by creating a pressure gradient between the lungs and the atmosphere.

As used herein, exhalation and expiration may be used interchangeably to mean the process of releasing air from the lungs through the nose or mouth.

As used in the description and claims, the singular form “a”, “an” and “the” include both singular and plural references unless the context clearly dictates otherwise. For example, the term “expandable member” may include, and is contemplated to include, a plurality of expandable members. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for modulating blood flow through a blood vessel, the method comprising:
implanting an occluding device in the blood vessel of a patient;
implanting a compressible bladder within a body cavity of the patient, the body cavity comprising a thoracic cavity or an abdominal cavity of the patient; and
cyclically compressing and decompressing the compressible bladder in response to pressure changes within the body cavity caused by breathing;
wherein the compressible bladder is mechanically or fluidly coupled to the occluding device such that compression and decompression of the compressible bladder cause the occluding device to vary a degree of occlusion of the blood vessel.

2. The method of claim 1, wherein:
the occluding device comprises a balloon, which is fluidly coupled to the compressible bladder by a conduit; and
wherein compression of the compressible bladder drives fluid into the balloon to at least partially inflate the balloon and increase occlusion of the blood vessel, and decompression of the compressible bladder withdraws fluid from the balloon to at least partially deflate the balloon and decrease occlusion of the blood vessel.

3. The method of claim 2, wherein the occluding device comprises a stent, wherein the balloon is disposed within the stent.

4. The method of claim 2, wherein:
the compressible bladder is implanted in the thoracic cavity;
pressure in the thoracic cavity increases during exhalation, which compresses the compressible bladder and drives fluid into the balloon to at least partially inflate the balloon and increase occlusion of the blood vessel; and
pressure in the thoracic cavity decreases during inhalation, which decompresses the compressible bladder and withdraws fluid from the balloon to at least partially deflate the balloon and decrease occlusion of the blood vessel.

5. The method of claim 2, wherein:
the compressible bladder is implanted in the abdominal cavity;
pressure in the abdominal cavity increases during inhalation, which compresses the compressible bladder and drives fluid into the balloon to at least partially inflate the balloon and increase occlusion of the blood vessel; and
pressure in the abdominal cavity decreases during exhalation, which decompresses the compressible bladder and withdraws fluid from the balloon to at least partially deflate the balloon and decrease occlusion of the blood vessel.

6. The method of claim 2, wherein the compressible bladder comprises a bistable mechanism configured to control the flow of the fluid between the compressible bladder and the balloon in response to pressure experienced by the bistable mechanism.

7. The method of claim 1, wherein:
the occluding device comprises an expandable member, which is coupled to the compressible bladder by a mechanical linkage; and
wherein compression and decompression of the compressible bladder causes movement of the mechanical linkage, which causes the expandable member to radially collapse or radially expand to vary the degree of occlusion of the blood vessel.

8. The method of claim 2, wherein the occluding device is implanted in the superior vena cava or the inferior vena cava.

9. The method of claim 1, wherein the compressible bladder is sutured to tissue in the body cavity.

10. A method of regulating blood flow in a patient, the method comprising:
implanting a compressible bladder within a body cavity of a patient such that the compressible bladder is exposed to cyclical pressure changes within the cavity during inhalation and exhalation, wherein the body cavity is a thoracic cavity or an abdominal cavity;
implanting an occluding device within a blood vessel, the occluding device including an expandable member, wherein the blood vessel is a superior vena cava or an interior vena cava;
fluidly or mechanically coupling the compressible bladder to the expandable member; and
allowing the cyclical pressure changes within the body cavity to actuate the compressible bladder, which in turn actuates the expandable member to modulate blood flow through the blood vessel.

11. The method of claim 10, wherein:
the expandable member is fluidly coupled to the compressible bladder by a conduit;
the cyclical pressure changes in the body cavity cause cyclical compression and decompression of the compressible bladder;
compression of the compressible bladder causes an inflation fluid to flow from the compressible bladder to the expandable member to expand the expandable member and increase occlusion of the blood vessel; and
decompression of the compressible bladder allows the inflation fluid to flow from the expandable member to the compressible bladder to allow the expandable member to constrict and decrease occlusion of the blood vessel.

12. The method of claim 11, wherein the expandable member comprises a self-expanding material and the compressible bladder comprises a self-constricting material such that the occluding device is biased to drive the inflation fluid from the compressible bladder to the expandable member.

13. The method of claim 11, wherein the expandable member comprises a self-constricting material and the compressible bladder comprises a self-expanding material such that the occluding device is biased to drive the inflation fluid from the expandable member to the compressible bladder.

14. The method of claim 11, wherein the compressible bladder is implanted in the thoracic cavity, wherein the method further comprises:
forcing the inflation fluid to flow from the compressible bladder into the expandable member via the conduit under an increase in pressure in the thoracic cavity during exhalation of the patient, which causes the expandable member to expand and increase occlusion of the blood vessel; and
allowing the inflation fluid to flow from the expandable member into the compressible bladder via the conduit under a decrease in pressure in the thoracic cavity during inhalation of the patient, which allows the expandable member to constrict and decrease occlusion of the blood vessel.

15. The method of claim 11, wherein the compressible bladder is implanted in the abdominal cavity, wherein the method further comprises:
forcing an inflation fluid to flow from the compressible bladder into the expandable member via the conduit under an increase in pressure in the abdominal cavity during inhalation of the patient, which causes the expandable member to expand and increase occlusion of the blood vessel; and
allowing the inflation fluid to flow from the expandable member into the compressible bladder via the conduit under a decrease in pressure in the abdominal cavity during exhalation of the patient, which allows the expandable member to constrict and decrease occlusion of the blood vessel.

16. The method of claim 11, wherein the occluding device comprises an annular stent anchored against an inner wall of the blood vessel, wherein the expandable member is coupled to the stent.

17. The method of claim 10, wherein the compressible bladder is mechanically connected to the expandable member by a mechanical linkage extending between the body cavity and the blood vessel.

18. The method of claim 17, wherein:
the expandable member comprises a membrane;
the cyclical pressure changes in the body cavity cause cyclical compression and decompression of the compressible bladder;
compression and decompression of the compressible bladder varies tension in the mechanical linkage, which causes the membrane to alternately collapse to increase occlusion of the blood vessel and expand to decrease occlusion of the blood vessel.

19. The method of claim 18, wherein the mechanical linkage comprises a flexible tension member.

20. A method of modulating venous blood flow in a patient, the method comprising:
implanting a compressible bladder within a thoracic cavity of the patient such that the compressible bladder is exposed to pressure changes in the thoracic cavity caused by respiration;
implanting an occluding device within a lumen of a vena cava of the patient, the vena cava comprising a superior vena cava or an inferior vena cava, the occluding device comprising a stent and an expandable member disposed within the stent, wherein the implanting the occluding device comprises anchoring the stent against an inner surface of the vena cava;
fluidly coupling the compressible bladder to the expandable member through a closed fluid pathway containing an inflation fluid; and
allowing respiratory-induced increases in pressure in the thoracic cavity to compress the compressible bladder and drive the inflation fluid into the expandable member to increase occlusion of the vena cava, and allowing respiratory-induced decreases in pressure in the thoracic cavity to permit decompression of the compressible bladder and withdrawal of the inflation fluid from the expandable member to decrease occlusion of the vena cava,
thereby cyclically modulating venous blood flow through the superior vena cava or the inferior vena cava in response to pressure changes in the thoracic cavity caused by respiration.
